(19) United States
(12) Patent Application Publication
(10) Pub. No.: US 2023/0095326 A1
(43) Pub. Date: Mar. 30, 2023

(54) ARTICULATED INSULATED COMPONENTS WITH ANGLED CORRUGATIONS

(71) Applicant: CONCEPT GROUP LLC, Wellesley, MA (US)

(72) Inventors: Shriram RADHAKRISHNAN, West Palm Beach, FL (US); Peter ROACH, Jacksonville, FL (US); David REYNOLDS, Deerfield Beach, FL (US)

(21) Appl. No.: 17/905,090

(22) PCT Filed: Feb. 26, 2021
(86) PCT No.: PCT/US2021/019872

§ 371 Date: Aug. 26, 2022

Related U.S. Application Data
(60) Provisional application No. 62/982,458, filed on Feb. 27, 2020.
(60) Provisional application No. 63/013,673, filed on Apr. 22, 2020.

(51) Int. Cl. F16L 59/14; F16L 59/065; F16L 11/15

(52) U.S. Cl. CPC F16L 59/143; F16L 59/065; F16L 11/15; A61M 25/0054

(57) ABSTRACT

Provided are thermally-insulated components that include a first tube enclosing a second tube, the first tube having individual corrugations or one or more helical or other corrugations. The corrugations can be non-perpendicular relative to the major axis of the tube, and the second tube and first tube can define a sealed insulating space of reduced pressure therebetween.

### RELATED APPLICATIONS

The present application claims priority to U.S. patent application No. 62/982,458, “Articulated Insulated Components” (filed Feb. 27, 2020) and U.S. patent application No. 63/013,673, “Articulated Insulated Components With Angled Corrugations” (filed Apr. 22, 2020). The foregoing applications are incorporated herein by reference in their entireties for any and all purposes.

### TECHNICAL FIELD

The present disclosure relates to the field of articulable thermally-insulated components.

### BACKGROUND

Existing insulated components (e.g., double-walled pipes) can be rigid and/or straight in form and configuration. This can limit their application, as such components cannot be bent or otherwise shaped after they are formed. This is a particular disadvantage in the medical instruments field, as medical instruments often need to be shapeable so as to facilitate their insertion into a patient as well as their use once inserted into the patient. This is also a disadvantage in the manufacturing field and other industrial fields, as it may be advantageous—or even necessary—to have insulating components (e.g., insulated conduits) that can be shaped so as to facilitate their assembly or incorporation into larger systems. It can also be advantageous to communicate fluid in a curved path within a confined space (e.g., a cabinet or compartment), especially where there is a need to communicate multiple fluids in a confined area while also maintaining the temperatures of those fluids. Accordingly, there is a need in the art for improved insulated components that can accommodate a curved fluid pathway within them, be changed in shape, and/or have other flexibility incorporated therein.

### SUMMARY

In meeting these described long-felt needs, the present disclosure first provides thermally-insulated devices, comprising: a first tube, the first tube defining a major axis and comprising a corrugated region that includes one or more corrugations, (a) the one or more corrugations defining a corrugation axis defined along the portion of the corrugation that is at the maximum distance measured radially outward from the major axis of the first tube, the corrugation axis being at a corrugation angle that is non-perpendicular to the major axis of the first tube, or (b) the corrugated region comprising a helical corrugation; and a second tube, the second tube having a major axis and the second tube being disposed within the first tube, and the second tube and first tube defining a sealed insulating space of reduced pressure therebetween.

Also provided are methods, comprising bending a device according to the present disclosure.

Further provided are methods, comprising bending a device according to the present disclosure.

Additionally provided are methods, comprising: with a device according to the present disclosure, communicating a fluid within the second tube.

Also disclosed are methods, comprising: with a device according to the present disclosure, communicating a fluid exterior to the first tube.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 provides an exterior view of an exemplary device;

FIG. 2 provides an exterior view of an exemplary device;

FIG. 3 provides an exterior view of an exemplary device;

FIG. 4 provides an exterior view of an exemplary device;

FIG. 5 provides an exterior view of an exemplary device;

FIG. 6 provides an exterior view of an exemplary device;

FIG. 7 provides an exterior view of an exemplary device;

FIG. 8 provides cross-sectional views of exemplary tubes;

FIG. 9 provides cross-sectional views of exemplary tubes

FIG. 10 provides cross-sectional views of exemplary tubes;

FIG. 11 provides cross-sectional views of exemplary tubes;

FIG. 12 provides cross-sectional views of exemplary tubes;

FIG. 13 provides cross-sectional views of exemplary tubes;

FIG. 14 provides cross-sectional views of exemplary tubes; and

FIG. 15 provides cross-sectional views of exemplary tubes.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable, and it should be understood that steps may be performed in any order.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. All documents cited herein are incorporated herein in their entireties for any and all purposes.

Further, reference to values stated in ranges include each and every value within that range. In addition, the term “comprising” should be understood as having its standard, open-ended meaning, but also as encompassing “consisting” as well. For example, a device that comprises Part A and Part B can include parts in addition to Part A and Part B, but can also be formed only from Part A and Part B.

### FIGURES

The attached figures are illustrative only and do not limit the scope of the present disclosure or the attached claims.

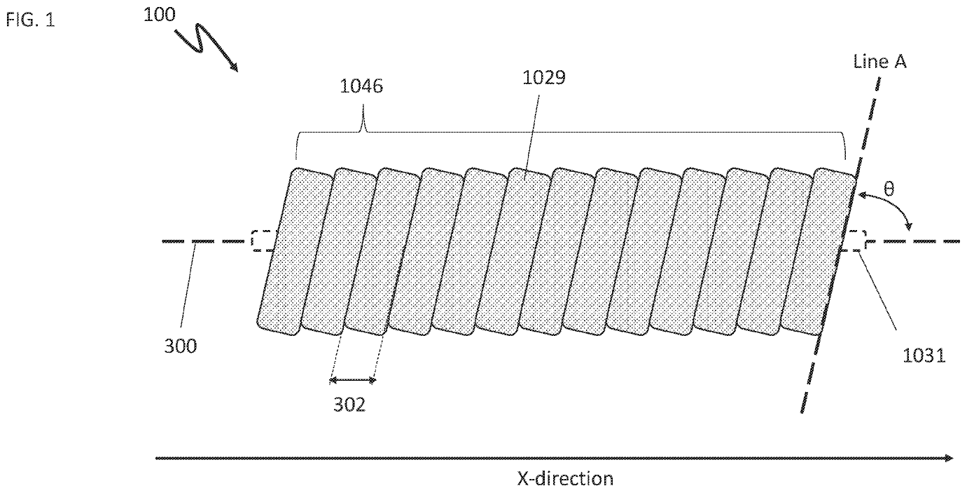

FIG. 1 provides a view of an exemplary device 100. As shown, first tube 1046 can include a plurality of (suitably parallel) corrugations 1029 along its length. As shown, the corrugations can define a direction that is disposed at an angle θ1 (along Line A) from major axis 300 of the first tube. Angle θ1 can be from −90 degrees to +90 degrees, preferably −45 degrees to +45 degrees. A corrugation can have a width 302. Corrugation width 302 can be variable along the length of tube 1046 (in the x-direction), but can also be constant. The pitch of the corrugations can be, e.g., from about 0.5 mm to about 5 mm, from about 1 mm to about 4.5 mm, from about 1.5 mm to about 4.0 mm, from about 2.0 mm to about 3.5 mm, or even about 2.5 mm. The corrugation pitch can be constant along the length of the tube 1046 (along the x-direction), but this is not a requirement, as the pitch can vary along the length of tube 1046 (in the x-direction). The pitch, height, and or spacing of the corrugations can be controlled to add differing degrees of flexibility to the insulating device as may be desired by the user. As an example, the corrugations along the bent portion of the tube can of a different pitch than the corrugations along a non-bent portion of the tube. Corrugations can be singular (e.g., circumferential ring-type corrugations that are perpendicular to the major axis of the tube), ring-type corrugations that are non-perpendicular to the major axis of the tube), but can also be helical in configuration. As an example, a component can include a helical corrugation that winds along some or all of a tube, similar to the helical stripe on a barber's pole.

Corrugations can be arranged (and the material of the tubes selected) so as to allow hand-bending of the devices, i.e., such that a person of ordinary size and strength could bend the device. This is not a requirement, however, as corrugations can be arranged (and materials of the tubes selected) such that a device is not hand-bendable and instead retains its shape (subject to changes caused by thermal stresses) unless bent by a machine.

It should be understood that a tube (whether the first tube or the second tube) can include corrugations along its entire length, but this is not a requirement. As an example, a tube can include corrugations along a middle portion of the tube's length but not at one or both ends of the tube. For example, the middle 1-99% of a tube's length can be corrugated, but the ends of the tube may be non-corrugated. As an example, in a 10 cm tube, the first 2 cm and the last 2 cm of the tube can be non-corrugated, with the middle 6 cm of the tube being corrugated. The uncorrugated portion of a tube can be used as a location for a joint (e.g., a joint between the first tube and the second tube).

Corrugations can be parallel to one another such that the “ridge” of a corrugation is parallel and distinct from the “ridge” of the adjacent corrugations. Corrugations can, however, also be in a spiral form, such that a single “ridge” (or multiple “ridges”) are formed circumferentially and longitudinally along the tube.

The width and/or pitch and/or height of corrugations can be varied along the length of a tube so as to modulate the rigidity of the tube at different locations along the tube's length. As an example, the width, height, and pitch of corrugations can be modulated such that the tube is relatively bendable around the middle of the tube and is relatively stiff at the ends of the tube. It should be understood that a tube can comprise corrugations along its entire length, but this is not a requirement, as a tube can comprise corrugations in one, two, three, or more separate regions. As an example, a tube can comprise two corrugated regions that are separated by a non-corrugated region.

A tube can also be telescoped (i.e., expanded) so as to change the tube's length, as well as the tube's rigidity. Corrugations can be formed so that the tube is bendable in only one direction, or so that the tube is preferentially bent in only one direction. A corrugation or corrugations can also be used as an expansion joint such that upon a change in temperature that effects an expansion (or contraction) of the tube's material, the corrugation expands or contracts thereby maintaining as constant the overall length and/or width of the tube.

Different parts of the tube(s) can be corrugated with different methods or directions to enhance flexibility of a particular segment(s) in one or all directions depending on the user's desires. For example, a tube can include a corrugated section and a non-corrugated section. In this way, a user can provide a tube that is flexible along the corrugated portion of its length but that is not flexible along its non-corrugated section. A component can include corrugations of different pitches and/or angles at different locations, which differently-configured corrugations can give rise to regions of different flexibility.

A component having corrugations that are oriented at an angle θ from major axis 300 can include any one or more of the other features described herein, e.g., conduit 1031, a pull wire, an aperture, and the like. A component can include corrugations that are all parallel to one another, but this is not a requirement, as a component can include two or more corrugations that are inclined at different angles (θ) from major axis 300.

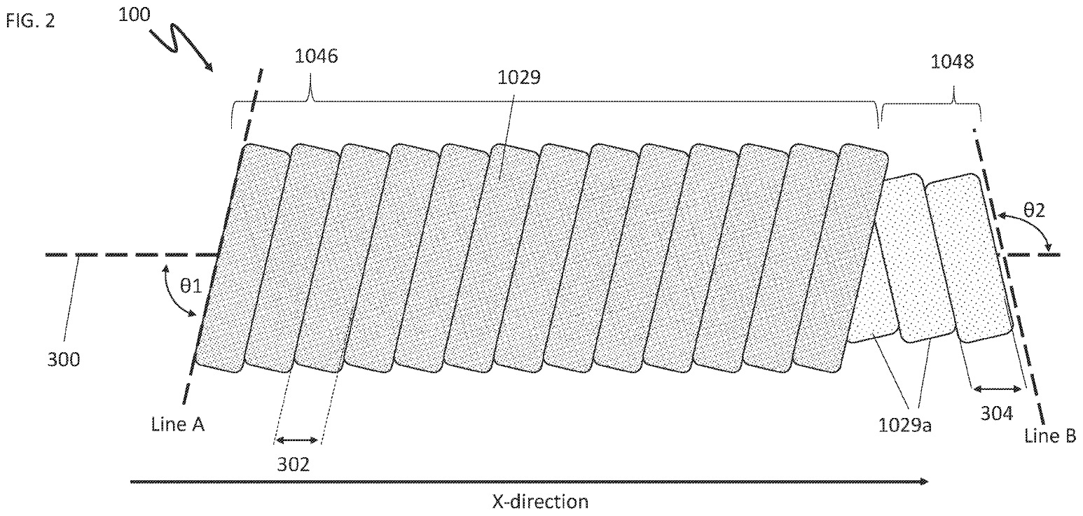

FIG. 2 provides a view of another embodiment of a component or device 100 according to the present disclosure. As shown, first tube 1046 can include a plurality of (parallel) corrugations 1029 along its length. As shown, the corrugations can be disposed at an angle θ1 from major axis 300 of the first tube. Angle θ1 can be from, e.g., −90 degrees to +90 degrees, preferably from −45 degrees to +45 degrees. A corrugation can have a width 302. Corrugation width 302 can be variable along the length of tube 1046 (in the x-direction), but can also be constant. As described elsewhere herein, the pitch of the corrugations in the first tube 1046 can be the same along the length of first tube 1046 (in the x-direction), but this is not a requirement, as the corrugation pitch can vary along the length of a tube.

Tube 1046 can enclose second tube 1048, which second tube can include corrugations 1029a, which corrugations are inclined at an angle θ2 from major axis 300 of the first tube. Angle θ2 can be from, e.g., −90 degrees to +90 degrees, preferably −45 degrees to +45 degrees. A corrugation can have a width 304. Corrugation width 304 can be variable along the length of tube 1048 (in the x-direction), but can also be constant. Corrugation width 304 can be, e.g, from about 0.5 mm to about 5 mm, or from about 1 mm to about 4.5 mm, or from about 1.5 mm to about 4.0 mm, or from about 2.0 mm to about 3.5 mm, or even from about 2.5 mm to about 3.0 mm. Similarly, the pitch of the corrugations in the second tube 1048 can be constant in the x-direction, but this is not a requirement, as the pitch of the corrugations in the second tube 1048 can vary in the x-direction. A sealed, evacuated space (described elsewhere herein) can be defined between first tube 1046 and second tube 1048. Corrugations 1029a can be parallel to one another, but individual corrugations 1029a can also be inclined at different angles from the major axis 300.

Corrugation width 304 of the corrugations of the second tube can be the same as corrugation width 302 of the first tube, but this is not a requirement. Corrugation width 304 of the corrugations of the second tube can be from 0.1 to 10 times the corrugation width 302 of the corrugations of the first tube.

Angle θ2 can be equal to 01, but this is not a requirement. θ2 can differ from θ1 by, e.g., from about 1 to about 89 degrees, from about 5 to about 85 degrees, from about 10 to about 80 degrees, from about 15 to about 75 degrees, from about 20 to about 70 degrees, from about 25 to about 65 degrees, from about 30 to about 60 degrees, from about 35 to about 55 degrees, or even from about 40 to about 50 degrees.

In addition, corrugations 1029a can also be of one or more spiral corrugations that, similar to the thread of a screw, extend around and along the length of second tube 1048. The pitch of such a spiral-type corrugation can be constant along the length of second tube 1048 (in the x-direction), but can also vary along the length of second tube 1048 (in the x-direction). One or both of first tube 1046 and second tube 1048 can comprise corrugations parallel to one another, and/or one or both of first tube 1046 and second tube 1048 can comprise corrugations that are spiral in configuration.

It should be understood that a corrugation can go completely around (i.e., 360 degrees) a tube, but this is not a requirement. A corrugation can go around only a portion of a tube's circumference, e.g., about 350 degrees, about 330 degrees, about 310 degrees, about 290 degrees, about 270 degrees, about 250 degrees, about 230 degrees, about 210 degrees, about 190 degrees, about 170 degrees, about 150 degrees, about 130 degrees, about 110 degrees, about 90 degrees, about 70 degrees, about 50 degrees, about 30 degrees, or even about 10 degrees.

Corrugations can go partially around the tube. For example, by reference to a clock dial, a tube can include a section that is corrugated from 12 to 3 o'clock and again from 6 to 9 o'clock. In this way, a tube can be constructed to bend in only one plane. Corrugations can also be uniform in depth, but can also be comparatively deep in depth (height) in one region (e.g., from 12 o'clock to 3 o'clock) relative to the corrugations at other regions about the time.

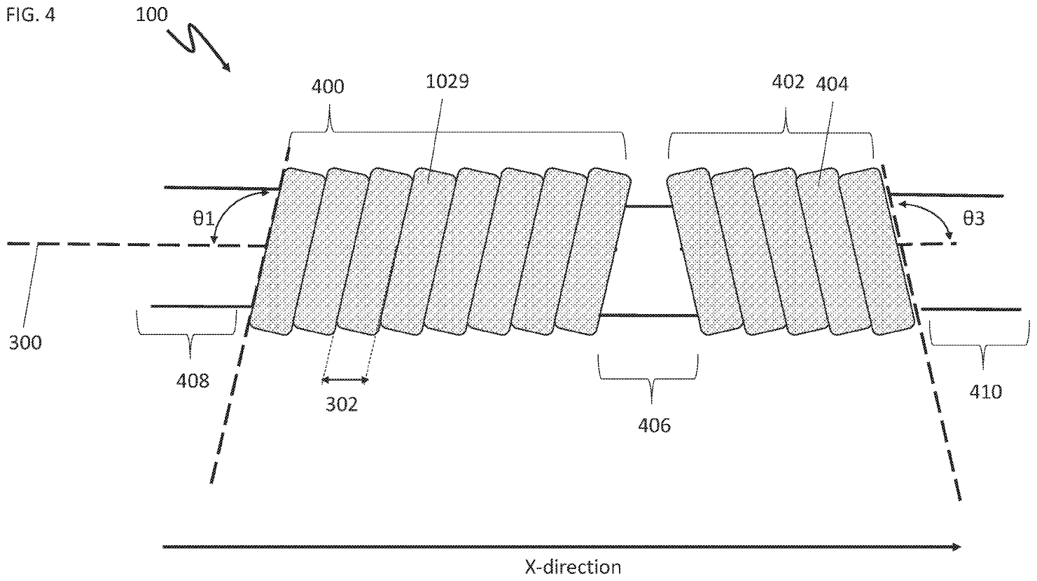

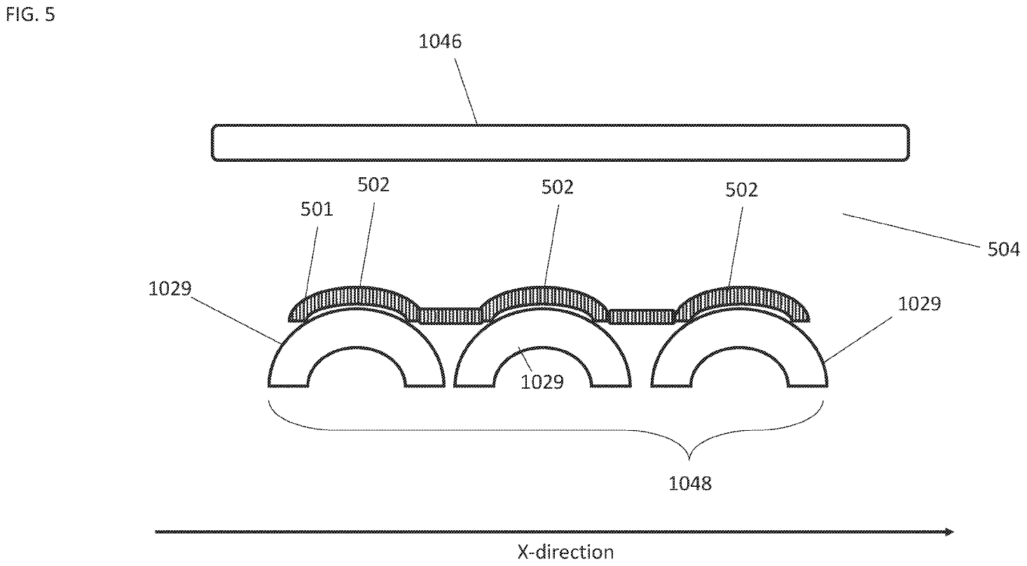

It should be understood that either one or both of first tube 1046 and second tube 1048 can be corrugated. It should also be understood that the corrugations in first tube 1046 can be inclined so as to “lean” in a direction that is different from the direction in which the corrugations in second tube 1048 “lean.” This is illustrated in FIG. 4, in which FIG. corrugations 1029 “lean” to the right, and corrugations 1029a “lean” to the left. It should be understood, however, that the first tube and the second tube can have corrugations that “lean” in the same direction. One of the first tube and the second tube can have corrugations that are perpendicular to the major axis 300, and the other of the first tube and the second tube can have corrugations that are non-perpendicular (whether parallel to one another or in spiral form) to the major axis 300.

Without being bound to any particular theory, the presence of corrugations that “lean” in different directions can improve bending in articles that are formed of multiple tubes. As an example, when the corrugations in an outer tube are canted differently than the corrugations

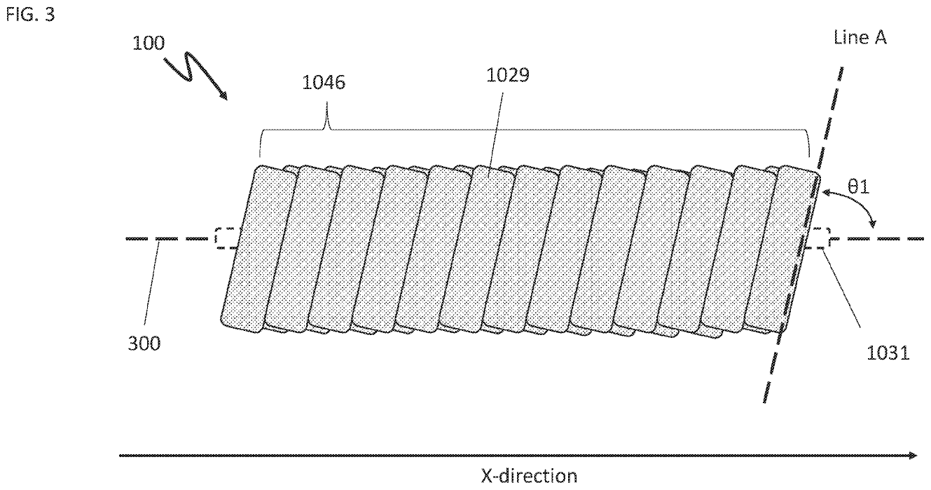

As shown in FIG. 3 (illustrating an exemplary device or component 100), corrugations 1029 can be of one or more spiral corrugations that, similar to the thread of a screw or the stripe on a barber pole, extend around and along the length of first tube 1046. The pitch of such a spiral-type corrugation can be constant along the length of second tube 1046 (in the x-direction), but can also vary along the length of second tube 1046 (in the x-direction). Such corrugations can define an angle θ1, as shown in FIG. 3.

FIG. 4 provides a further exemplary embodiment of a device or component 100 according to the present disclosure. As shown, a tube (not labeled) can include a first region 400 having corrugations 1029 thereon that are angled at an angle θ1 relative to the centerline 300 of the tube. The tube can include a second region 402 having corrugations 404 thereon that are angled at an angle θ3 relative to the centerline 300. As shown, the tube can also include a region 406 that is uncorrugated, which region can connect corrugated regions 400 and 404. A tube can also include uncorrugated sections at the ends (shown by 408 and 410); an uncorrugated section need not necessary be positioned to bridge two corrugated sections.

Angle θ1 can differ from θ3 by from about 1 to about 90 degrees, e.g., from about 1 to about 89 degrees, from about 5 to about 85 degrees, from about 10 to about 80 degrees, from about 15 to about 75 degrees, from about 20 to about 70 degrees, from about 25 to about 65 degrees, from about 30 to about 60 degrees, from about 35 to about 55 degrees, or even from about 40 to about 50 degrees. It should be understood that a given tube can have 0 corrugated regions, 1 corrugated region, or multiple corrugated regions.

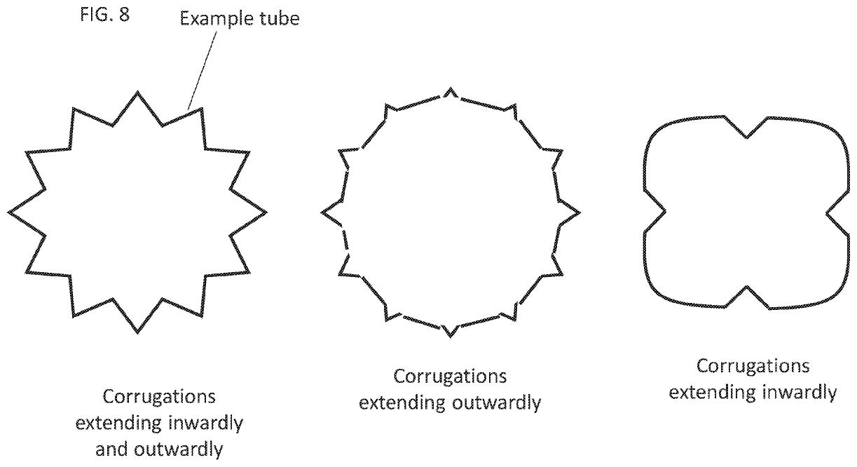

It should also be understood that corrugations can extend outwardly from the outer surface of a tube, extend inwardly from the inner surface of a tube, or extend outwardly and inwardly from the tube. As one example, a threading die can be used to establish the corrugations. Example such corrugations are shown in FIG. 8, which figure shows a tube with corrugations extending inwardly and outwardly (left panel), corrugations extending outwardly (middle panel), and corrugations extending inwardly (right panel). A corrugation can be in the form of a ridge with a sharp V-peak, but can also be in the form of a ridge with a rounded U-peak. As shown by FIG. 8, tubes need not necessarily be circular in cross-section; a tube can be polygonal, ovoid, star-shaped, circular with flat areas, or otherwise shaped according to the user's needs.

The width of the corrugations (illustrated by 302) can be constant, but individual corrugations can have different widths and/or pitches. It should also be understood that corrugations can be parallel to one another, but a tube can include one or more regions where the corrugations are in spiral form, e.g., a region with a single spiral corrugation that runs along a region of the tube. The configuration shown in FIG. 4 can be utilized on an outer tube that encloses an inner tube, but can also be used on an inner tube that is enclosed within an outer tube.

A further embodiment is shown in FIG. 7. As shown in that figure, a component 100 can include a second tube 1048 (the first tube of the component is not shown), which second tube 1048 includes a plurality of corrugations 1029a, as shown. The corrugations can be inclined at an angle θ4, measured as the angle of Line C relative to the major axis (not shown) of second tube 1048. The corrugations can have a width 304.

A spacer material 702 can be present. As described elsewhere herein, spacer material 702 can be in thread form; it can also be in the form of stripes, a sleeve, a sheet, and the like. Material in the form of a sheet can have the edges of the sheet secured to one another so as to form a sleeve. The spacer material can be wound about tube 1048 such that the spacer material lies across the peaks/valleys of corrugations 1029a. The spacer material can be secured in place by a further material 704, which further material can be threads, stripes, or other forms of material. The presence of the further material is not a requirement. The further material can be wound or otherwise placed so that it is perpendicular to the spacer material. The further material can also be wound or otherwise placed so that it is non-perpendicular to the spacer material, and can even be wound or otherwise placed so that it is parallel to the spacer material. Although not shown in the appended figures, a device can include a further sheathing material, which sheathing material can be disposed between the spacer material and a tube.

Although not shown in the figures, a component according to the present disclosure can include a linkage (e.g., a linkage in communication with an actuator or controller) that is configured to deflect a portion of the component, e.g., to rotate and/or bend a portion of the component. The linkage can be removeable.

Exemplary walls, sealing processes, and insulating spaces can be found in, e.g., US2018/0106414; US2017/0253416; US2017/0225276; US2017/0120362; US2017/0062774; US2017/0043938; US2016/0084425; US2015/0260332; US2015/0110548; US2014/0090737; US2012/0090817; US2011/0264084; US2008/0121642; US2005/0211711; WO/2019/014463; WO/2019/010385; WO/2018/093781; WO/2018/093773; WO/2018/093776; PCT/US2018/047974; WO/2017/152045; U.S. 62/773,816; and U.S. Pat. No. 6,139,571, the entireties of which documents are incorporated herein for any and all purposes.

A device can be configured such that the distal end of the device is configured for insertion into a subject. The distal end can be rounded, pointed, cupped, or otherwise shaped to support the needs of the user.

The device or a portion thereof is adapted to be inserted into a patient's body by way of a natural access (e.g., the nose, mouth, or rectum) or by a small incision in the body (e.g., into anatomical locations such as the pleural cavity, abdomen, or neck). In some embodiments, the device include a channel or other guide for permitting the passage of a surgical accessory implement (such as a cutting tool, a telescope, a manipulator, a secondary aspiration and irrigation device, etc.) into a surgical site.

A device can be configured for passage of fluid pumped from a remote fluid source ultimately to a surgical site. An irrigation control valve assembly operatively associated with the handle can allow the user to selectively modulate the flow of fluid through at least one of a first and a second fluid flow path. Fluid can be provided from a remote source, but a device according to the present disclosure can include a fluid source, e.g., a reservoir. In one embodiment, the device comprises a fluid flow control valve assembly that allows the user to selectively direct the flow of fluid. For example, the assembly can allow fluid to pass along a path that heats and/or cools the fluid and then passes the fluid to a surgical site to effect a therapeutic result. The assembly can allow fluid to pass along a path that sends the fluid directly to a surgical site without affirmative heating and/or cooling.

A device according to the present disclosure can include a heater and/or chiller configured to supply (or remove) heat from fluid passing within the device.

A device can include a temperature controller that receives fluid temperature information sensed by one or more temperature sensors disposed in or on the device.

In an alternative embodiment of the present invention, the device comprises an aspiration train, which train can include valves, tubing, a source of pressure (and/or of negative or reduced pressure) and the like. The aspiration train permits the user to control the suction pressure to effect withdrawal of fluid or friable tissue from the surgical site.

In some embodiments, a material (which can be termed a spacer material) is disposed between the first tube and second tube, within the sealed insulating space between the tubes. The material can be in the form of a sheet, in woven form, in non-woven form, in fibrous form, in a porous form, in a perforated form, in a mesh form, in strip form, in thread form (e.g., ceramic threads), and the like. The material can be disposed so as to reduce or eliminate physical contact between the first and second walls. The material can also itself be corrugated, crinkled, embossed, or include other surface features that act to minimize the contact area between the material and the tubes.

The material can be configured so as to lie in register with one or more corrugations of the tubes. In this way, the material can remain in position once installed between the tubes. As an example, the material can be a concave tape in form, which concave tape is wound about the peaks of the corrugations of the inner (second) tube such that the tape is maintained in position. A tape can also be wound about the peaks of the corrugations of the outer tube.

An example of the foregoing is shown in non-limiting FIG. 5. As shown in that figure, spacer material 501 is disposed within the evacuated space 504 between the first tube 1046 and the second tube 1048, and the material can include ridges 502, which ridges are wound about the peaks of the corrugations 1029 of second tube 1048. In non-limiting FIG. 5, first tube 1048 does not include any corrugations. (It should be understood that in the disclosed components, either of the first tube and the second tube can be partially or totally free of corrugations. Without being bound to any particular theory, this can provide an analogue to an expansion joint in which the thermal expansion of a tube is accommodated by the corrugations of that tube. In this way, a tube can maintain its length when heated, and the corrugations of the tube can take up any mechanical stress that can arise from the heating. The ridges 502 of the material in turn maintain the material in place and can reduce the material sliding out of position during use.

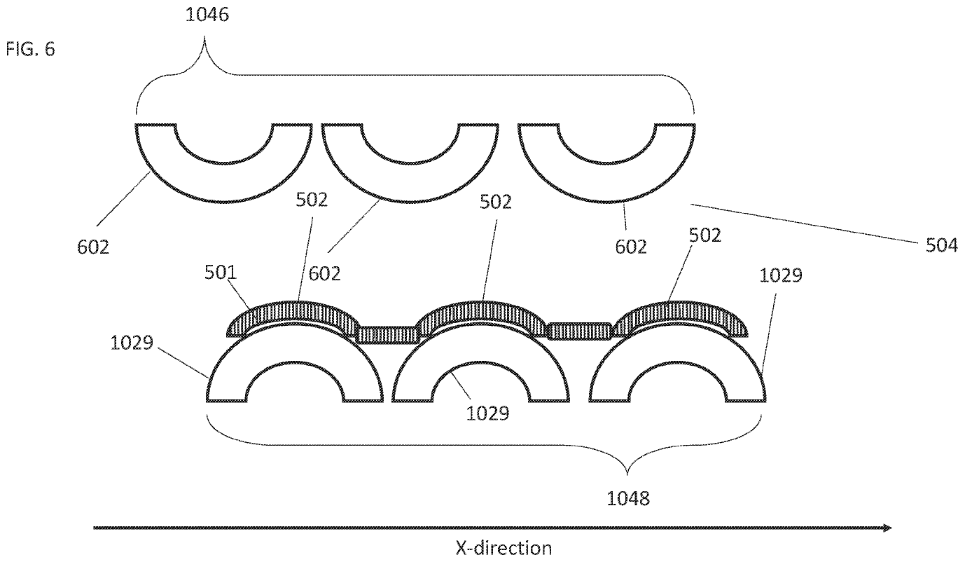

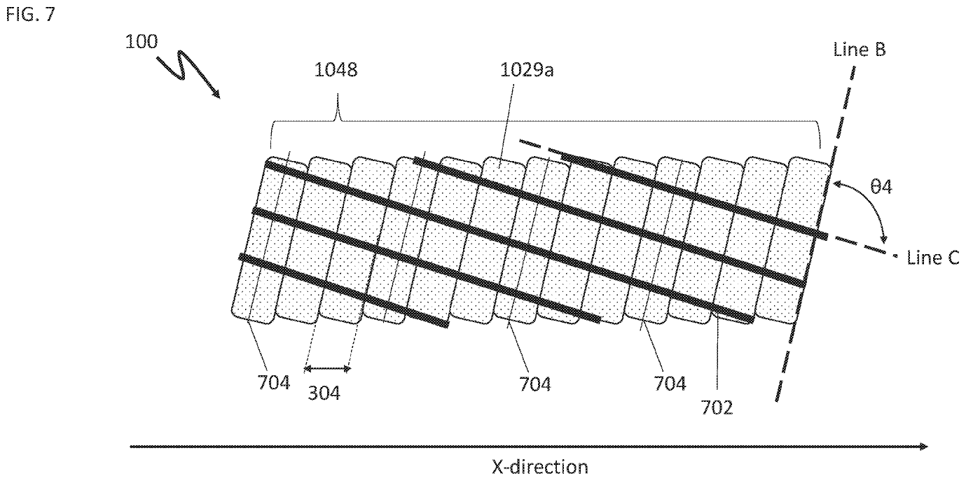

Non-limiting FIG. 6 provides a similar configuration as FIG. 5; as shown in FIG. 6, first tube 1046 can include corrugations 602.

The material can be, e.g., a multi-layer material, e.g., a material that comprises multiple layers of thin sheets. Such material can comprise materials that are made of, e.g., plastic, such as Mylar™ or Kapton™, coated on one, or both sides with a thin layer of metal, typically silver or aluminum. The material can also be metal, i.e., be a sheet of metal, such as silver, aluminum, copper, or other metal or alloy. Such layers in the multi-layer material can be spaced closely together, but can be free of contact from one another and separated by, e.g., a mesh, scrim, or other material. The multi-layer material can be configured so as to minimize thermal conduction between the layers. The multi-layer material can include layers that are embossed, crinkled, folded, peaked, or otherwise include surface features so as to reduce contact between layers and/or to reduce contract between the multilayer material and the tubes. A multilayer material can be reflective; as an example, such a material can have an emissivity of less than about 0.4, 0.3, 0.2, 0.15, 0.1, 0.09, 0.08, 0.07, 0.06, 0.05, 0.04, 0.03, 0.02, or even lower for wavelengths in the range of from about 0.02 microns to about 50, to about 45, to about 40, to about 35, to about 30, to about 25, or even to about 15 or 16 micrometers.

As an example, a material can include criss-crossing folds, ridges, or other surface features. Such features can in turn lie within the valleys of the corrugations of the tubes between which the material is disposed. In this way, the surface features of the material act to maintain the material in position and/or configuration and can reduce or even prevent the material from sliding out of place.

An example is useful to illustrate this configuration. By reference to FIG. 2, a component can include an outer (first) tube 1046 with corrugations 1029, which corrugations are angled at an angle θ1 of 45° relative to the major axis 300 of the tube. The component can further include an inner (second) tube 1048 that has corrugations 1029a that are angled at an angle θ2 that is 90° from 01. A spacer material can be disposed between the first tube and the second tube, the spacer material having criss-crossing wrinkles that are of a pitch such that the peaks of the wrinkles lie along the valleys of the corrugations of the first tube and the second tube.

Corrugations can be annular; corrugations can also be spiral. Corrugations can be periodic, but this is not a requirement, as corrugations can be disposed in a non-periodic fashion. The first tube and the second tube can both be corrugated, though this is not a rule or requirement. The first tube and the second tube can bear the same type of corrugations (e.g., corrugations having the same height and the same period), but this is not a requirement. The second tube can have a corrugated region that is in at least partial register with a corrugated region of the first tube, but this also is not a requirement. A tube can include one, two, or more corrugated regions; corrugated regions can be separated from one another by non-corrugated regions.

In some embodiments, a device according to the present disclosure can include a heater in the inner tube of the device, i.e., located within the. A heater can comprise a lead, which lead can be flexible. A lead can extend through the sealed insulating space between the inner tube of the device and the outer tube of the device. Alternatively, a lead can extend into the lumen of the device into an end of the lumen, i.e., without passing through the sealed insulating space between the inner tube of the device and the outer tube of the device.

A fluid (e.g., water, air) can be passed over the heater such that the fluid is heated. In some embodiments, the heater can be configured to give rise to steam within the lumen of the device, which steam can be communicated along the lumen. In some embodiments, a plug or check valve can be installed on one end of the device therefore causing the pressure developed by the production of steam to force the fluid or steam in one direction through the device.

A heater can be, e.g., tubular in shape, cylindrical in shape, finned, or of other configuration. A device according to the present disclosure can comprise a single heater or a plurality of heaters. In some embodiments, the heater can be extended and/or withdrawn from the lumen of the device. Without being bound to any particular embodiment or theory of operation, a heater can be configured as a spike or probe that is extended and/or withdrawn into the lumen. A heater can be a resistance heater, e.g., a heater in which the passage of an electric current through a heating element produces heat. Alternatively, the heater can be an induction heater. In some such embodiments, an electrically conductive and magnetically permeable heating element or susceptor (e.g., a wire) is heated by action of eddy currents produced by an electronic oscillator (e.g., a coil) that gives rise to the currents in the susceptor. One or both of the susceptor and the electronic oscillator can be moveable relative to the other. As an example, the susceptor can be extendable into the lumen of the device such that the susceptor experiences a certain level of heating as a result of eddy currents produced by a coil of the device. In addition or alternatively, the coil can be moveable.

A coil can be disposed within the lumen of the device, e.g., within the inner wall of the device. The coil can be configured such that fluid communicated within the lumen contacts the coil, although this is not a requirement. A coil can also be disposed within the sealed insulating space of the device, e.g., between the inner wall and the outer wall of the device. A coil can also be disposed exterior to the outer wall of the device

A device according to the present disclosure can include, e.g., a temperature probe. Such a probe can be configured to measure a temperature within the lumen of the device, e.g., the temperature of a fluid disposed within the lumen. The temperature probe can also be configured to measure the temperature of a susceptor, a wall of the lumen, or other element of the device. It should also be understood that a heater can be used as a temperature monitoring device; this can be accomplished by testing the resistance of the heater to determine the temperature of the heater and/or the fluid being heated.

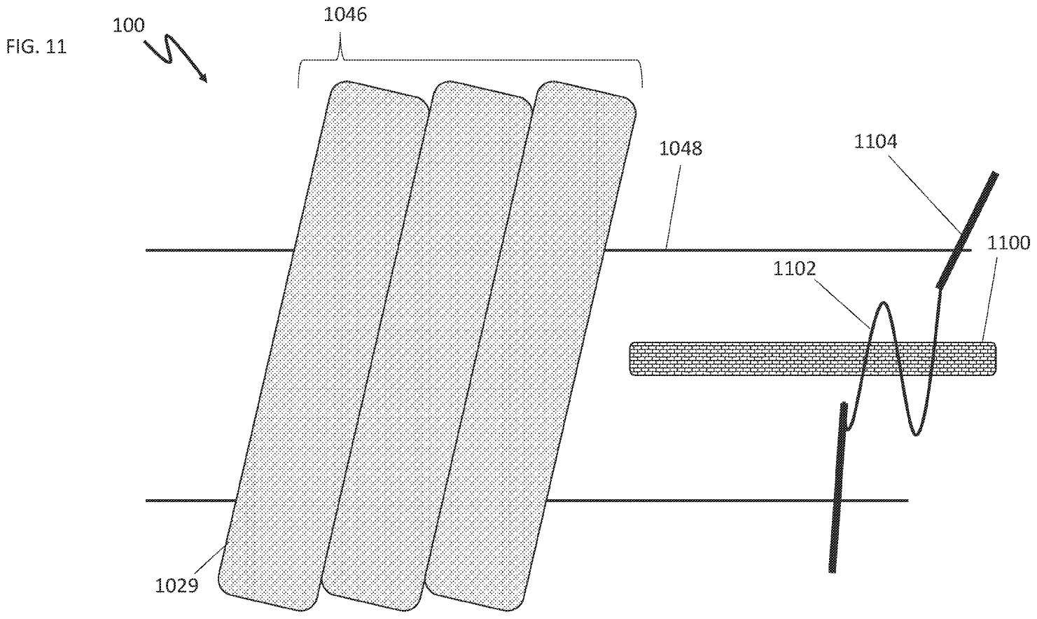

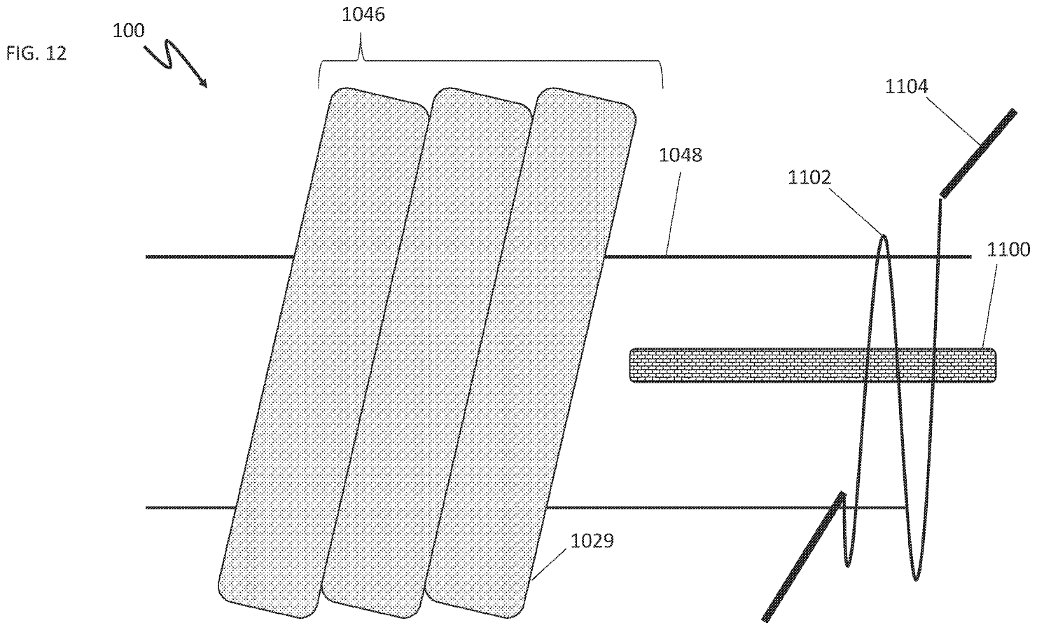

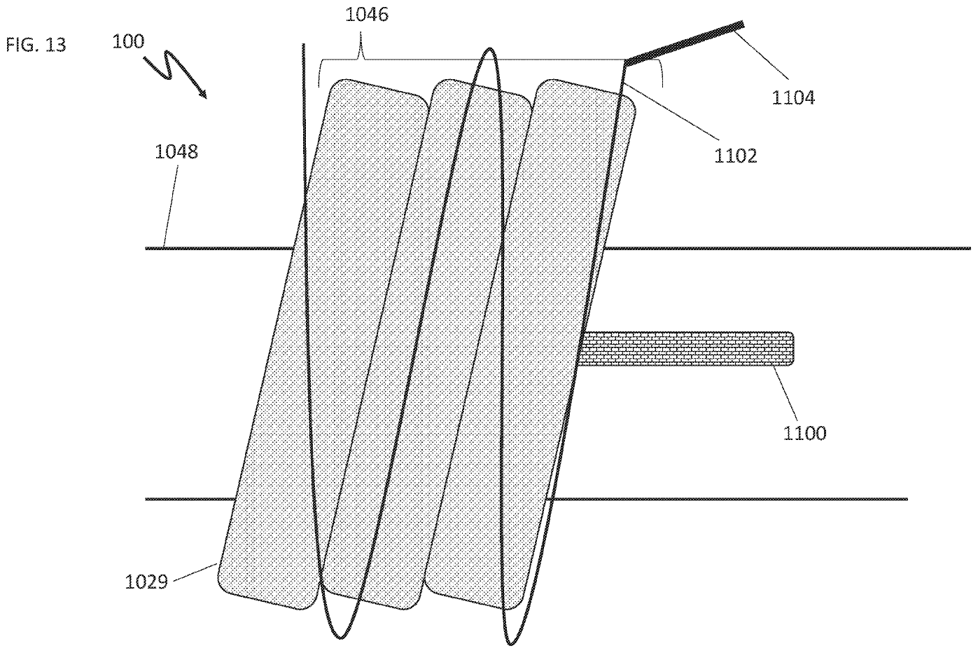

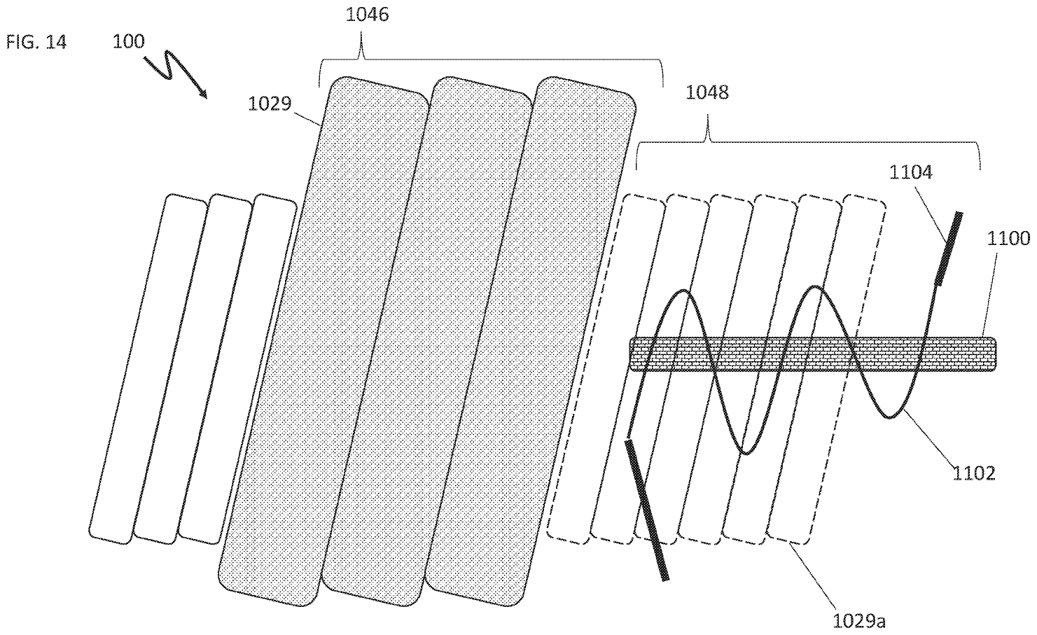

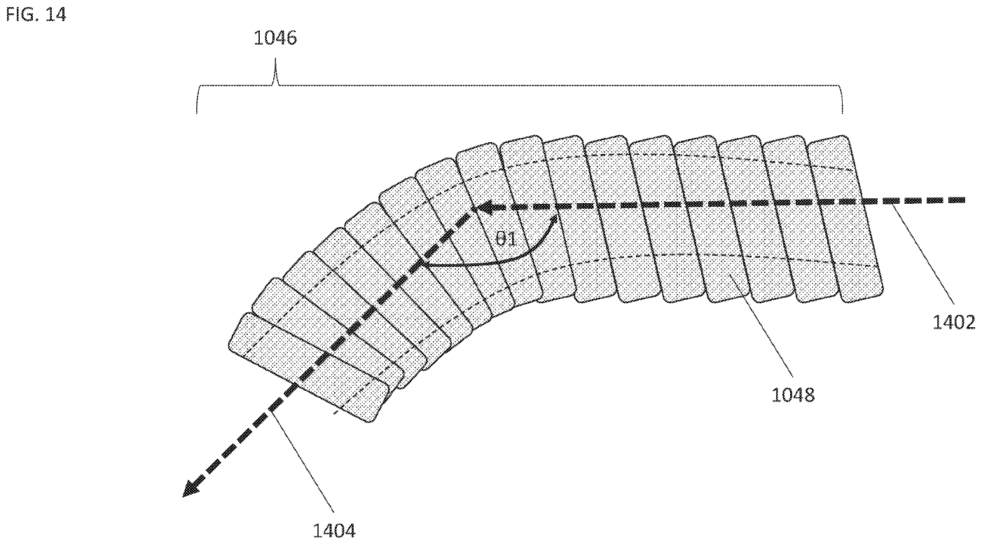

Example embodiments of the foregoing are shown in FIG. 11, FIG. 12, FIG. 13, and FIG. 14. As shown in FIG. 11 (illustrating example device 100), a first tube having corrugations 1029 is positioned above second tube 1048 so as to define a sealed insulating space between the first (outer) and second (inner) tubes. A susceptor 1100 can be position within second tube 1048; as described elsewhere herein, the susceptor can be of a material (e.g., a ferrous metal) that is susceptible to inductive heating. An inductive heating coil 1102 can be used to effect the inductive heating of susceptor 1100. As shown, inductive heating coil 1102 can include one or more leads 1104. A lead can extend through tube 1048 and into the sealed insulating space between tube 1048 and second tube 1046. Tube 1048 can include corrugations, which corrugations are for clarity not shown in FIG. 11.)

As described elsewhere herein, susceptor 1100 can be a wire. This is not a requirement, however, as a susceptor can be a rod or of any other shape. A susceptor can extend in the direction of the major axis of the lumen, but this too is not a requirement, as a susceptor can extend into the lumen, e.g., perpendicularly to the major axis of the lumen. A susceptor can extend at an angle (e.g., from 1 degree to 89 degrees) to the major axis of the lumen. The susceptor can be moveable, e.g., can be extended and/or withdrawn into the lumen.

Similarly, the inductive heating coil 1102 can also be moveable. The heating coil can be extended into (or along) the lumen of the device. A heating coil can be located within the lumen (e.g., to encircle a susceptor), but this is not a requirement, as a heating coil can be located in the insulating space between the first tube and second tube, or even located exterior to the outermost tube.

It should be understood that a device can include one susceptor or even a plurality of susceptors. Individual susceptors can be fixed, but can also be independently moveable or moveable in a group or in groups. Likewise, a device can include one heating coil or a plurality of heating coils. Individual coils be fixed, but can also be independently moveable or moveable in a group or in groups.

FIG. 12 provides a view of an alternative embodiment of the disclosed devices. As shown in FIG. 12 (illustrating example device 100), a first tube 1046 having corrugations 1029 is positioned above second tube 1048 so as to define a sealed insulating space between the first (outer) and second (inner) tubes. A susceptor 1100 can be position within second tube 1048; as described elsewhere herein, the susceptor can be of a material (e.g., a ferrous metal) that is susceptible to inductive heating. An inductive heating coil 1102 can be used to effect the inductive heating of susceptor 1100. As shown, inductive heating coil 1102 can include one or more leads 1104. A lead can extend through tube 1048 and into the sealed insulating space between tube 1048 and second tube 1046. Tube 1048 can include corrugations, which corrugations are for clarity not shown in FIG. 12.) As shown in FIG. 12, heating coil 1102 can at least partially encircle second tube 1048.

FIG. 13 provides a view of an alternative embodiment of the disclosed devices. As shown in FIG. 13 (illustrating example device 100), a first tube 1046 having corrugations 1029 is positioned above second tube 1048 so as to define a sealed insulating space between the first (outer) and second (inner) tubes. A susceptor 1100 can be position within second tube 1048; as described elsewhere herein, the susceptor can be of a material (e.g., a ferrous metal) that is susceptible to inductive heating. An inductive heating coil 1102 can be used to effect the inductive heating of susceptor 1100. As shown, inductive heating coil 1102 can include one or more leads 1104. A lead can extend through tube 1048 and into the sealed insulating space between tube 1048 and second tube 1046. A lead can also extend through first tube 1046. Tube 1048 can include corrugations, which corrugations are for clarity not shown in FIG. 13.) As shown in FIG. 12, heating coil 1102 can at least partially encircle first tube 1046.

FIG. 14 provides a view of an alternative embodiment of the disclosed devices. As shown in FIG. 14, a first tube 1046 having corrugations 1029 is positioned above second tube 1048 (having corrugations 1029a) so as to define a sealed insulating space between the first (outer) and second (inner) tubes. A susceptor 1100 can be position within second tube 1048; as described elsewhere herein, the susceptor can be of a material (e.g., a ferrous metal) that is susceptible to inductive heating.

An inductive heating coil 1102 can be used to effect the inductive heating of susceptor 1100. As shown, inductive heating coil 1102 can include one or more leads 1104. A lead can extend through tube 1048 and into the sealed insulating space between tube 1048 and second tube 1046. A lead can also extend through first tube 1046. As shown in FIG. 14, heating coil 1102 can be disposed within second tube 1048.

Although not shown in FIGS. 11-14, devices according to those figures can include a spacer material disposed between the first tube and the second tube.

As described elsewhere herein, the disclosed devices can be flexible, which flexibility facilitates their incorporation into various applications where a curved fluid pathway can be useful. As one example, the disclosed devices can be used to dispense steam, hot milk, irrigation fluid (e.g., saline), and the like. The disclosed devices can also be shaped so as to facilitate their assembly or incorporation into larger systems, in particular systems (e.g., automotive, aerospace, naval) where curved fluid pathways are needed in confined spaces. As an example, the disclosed devices have particular application in automotive applications, where there may be a need to convey a comparatively cool fluid in close proximity to a comparatively hot fluid, while maintaining the temperatures of the two fluids.

A device according to the present disclosure can define within it a linear flow path (e.g., the pathway defined by the lumen of the second/inner tube), but this is not a requirement. A device according to the present disclosure can define a flow path that is C-shaped, V-shaped, S-shaped, Z-shaped, or otherwise non-linear. A device can define a first flow path portion and a second flow path portion, with direction of flow within the first flow path portion and the second flow path portion being from about 1 to about 180 degrees, e.g., from about 1 to about 180 degrees, from about 5 to about 175 degrees, from about 10 to about 170 degrees, from about 15 to about 165 degrees, from about 20 to about 160 degrees, from about 25 to about 160 degrees, from about 30 to about 155 degrees, from about 35 to about 150 degrees, from about 40 to about 145 degrees, from about 45 to about 140 degrees, from about 50 to about 135 degrees, from about 55 to about 130 degrees, from about 60 to about 125 degrees, from about 65 to about 120 degrees, from about 70 to about 115 degrees, from about 75 to about 110 degrees, from about 80 to about 105 degrees, from about 85 to about 100 degrees, or even from about 90 to about 95 degrees.

An example of this is shown by non-limiting FIG. 14 (illustrating example device 100). As shown in FIG. 14, first (outer) tube 1046 can be disposed about second (inner) tube 1048. First tube 1046 can include corrugations (shown but not labeled); similarly, second tube 1048 can include corrugations (not shown). As shown, component or device 100 can define a first flow path region 1402 extending within the second tube and a second flow path region 1404 extending within the second tube. First flow path region 1402 and second flow path region 1404 can be at an angle θ1 to one another. Angle θ1 can be from about 1 to about 180 degrees, e.g., from about 1 to about 180 degrees, from about 5 to about 175 degrees, from about 10 to about 170 degrees, from about 15 to about 165 degrees, from about 20 to about 160 degrees, from about 25 to about 160 degrees, from about 30 to about 155 degrees, from about 35 to about 150 degrees, from about 40 to about 145 degrees, from about 45 to about 140 degrees, from about 50 to about 135 degrees, from about 55 to about 130 degrees, from about 60 to about 125 degrees, from about 65 to about 120 degrees, from about 70 to about 115 degrees, from about 75 to about 110 degrees, from about 80 to about 105 degrees, from about 85 to about 100 degrees, or even from about 90 to about 95 degrees. Although not shown in FIG. 14, a device can define a third flow path portion, which third flow path portion can be at an angle of from about 1 to about 180 degrees, e.g., from about 1 to about 180 degrees, from about 5 to about 175 degrees, from about 10 to about 170 degrees, from about 15 to about 165 degrees, from about 20 to about 160 degrees, from about 25 to about 160 degrees, from about 30 to about 155 degrees, from about 35 to about 150 degrees, from about 40 to about 145 degrees, from about 45 to about 140 degrees, from about 50 to about 135 degrees, from about 55 to about 130 degrees, from about 60 to about 125 degrees, from about 65 to about 120 degrees, from about 70 to about 115 degrees, from about 75 to about 110 degrees, from about 80 to about 105 degrees, from about 85 to about 100 degrees, or even from about 90 to about 95 degrees to the second flow path portion.

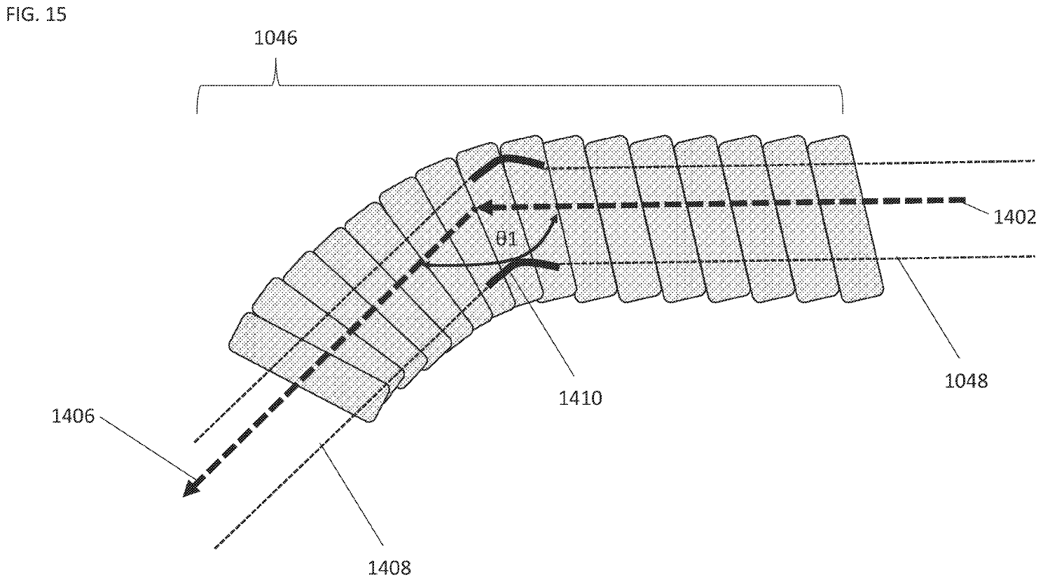

Another example is provided by non-limiting FIG. 15 (illustrating example device 100). As shown in FIG. 14, first (outer) tube 1046 can be disposed about second (inner) tube 1048 and third tube 1408. First tube 1046 can include corrugations (shown but not labeled); similarly, second tube 1048 can include corrugations (not shown). Similarly, third tube 1408 can include corrugations (not shown). As shown, second tube 1048 and third tube 1408 can be joined by fitting 1410, which fitting can be an elbow joint or other such fitting. Second tube 1048 and third tube 1408 can also be joined directly to one another (not shown).

As shown, component or device 100 can define a flow path region 1402 extending within the second tube and a flow path region 1406 extending within the third tube. First flow path region 1402 and second flow path region 1406 can be at an angle θ1 to one another. Angle θ1 can be from about 1 to about 180 degrees, e.g., from about 1 to about 180 degrees, from about 5 to about 175 degrees, from about 10 to about 170 degrees, from about 15 to about 165 degrees, from about 20 to about 160 degrees, from about 25 to about 160 degrees, from about 30 to about 155 degrees, from about 35 to about 150 degrees, from about 40 to about 145 degrees, from about 45 to about 140 degrees, from about 50 to about 135 degrees, from about 55 to about 130 degrees, from about 60 to about 125 degrees, from about 65 to about 120 degrees, from about 70 to about 115 degrees, from about 75 to about 110 degrees, from about 80 to about 105 degrees, from about 85 to about 100 degrees, or even from about 90 to about 95 degrees. Although not shown in FIG. 14, a device can define a third flow path portion, which third flow path portion can be at an angle of from about 1 to about 180 degrees, e.g., from about 1 to about 180 degrees, from about 5 to about 175 degrees, from about 10 to about 170 degrees, from about 15 to about 165 degrees, from about 20 to about 160 degrees, from about 25 to about 160 degrees, from about 30 to about 155 degrees, from about 35 to about 150 degrees, from about 40 to about 145 degrees, from about 45 to about 140 degrees, from about 50 to about 135 degrees, from about 55 to about 130 degrees, from about 60 to about 125 degrees, from about 65 to about 120 degrees, from about 70 to about 115 degrees, from about 75 to about 110 degrees, from about 80 to about 105 degrees, from about 85 to about 100 degrees, or even from about 90 to about 95 degrees to the second flow path portion. A user can effect communication of a fluid within the second tube and the third tube.

### EMBODIMENTS

The following non-limiting embodiments are illustrative only and do not serve to limit the scope of the present disclosure or of the attached claims.

Embodiment 1. A thermally-insulated device, comprising: a first tube, the first tube defining a major axis, the first tube comprising a corrugated region that includes one or more corrugations, (a) the one or more corrugations defining a corrugation axis defined along the portion of the corrugation that is at the maximum distance measured radially outward from the major axis of the first tube, the corrugation axis being at a corrugation angle that is non-perpendicular to the major axis of the first tube, or (b) the corrugated region comprising a helical corrugation; and a second tube, the second tube having a major axis and the second tube being disposed within the first tube, and the second tube and first tube defining a sealed insulating space of reduced pressure therebetween.

As shown and described herein, corrugations can be in a helical form. Alternatively, corrugations can be in the form of rings or revolved semicircles that are parallel to one another, e.g., as a row of rings positioned next to one another. (A ring can have the form of a revolved semicircle or arch.) As shown in FIG. 1, corrugations 1029 are present as a parallel set of rings. As shown in FIG. 3, corrugations 1029 are present in a helical form.

As described elsewhere herein, a device according to the present disclosure can include a susceptor or resistance heater (or both), which susceptor or heater can be used to heat a fluid disposed within the second tube. In some embodiments, however, the second tube can itself be a susceptor, and the device can be arranged such that a heating coil gives rise to inductive heating of the second tube.

Embodiment 2. The device of Embodiment 1, wherein the second tube comprises a corrugated region that includes one or more corrugations, (a) the one or more corrugations defining a corrugation axis defined along the portion of the corrugation that is at the maximum distance measured radially outward from the major axis of the second tube, the corrugation axis being at a corrugation angle that is non-perpendicular to the major axis of the second tube, or (b) the corrugated region of the second tube comprising a helical corrugation. The corrugations of the first tube and the second tube can be angled in the same direction, but can also be angled against each other, as shown in FIG. 2. Without being bound to any particular theory, corrugations of the first tube and the second tube in the same direction can facilitate bending in one direction. If the corrugations of the first tube and second tube are in a counter rotating direction, the component may bend in a predictable manner in all directions.

Embodiment 3. The device of Embodiment 2, wherein the one or more corrugations of the second tube run in a direction different from a direction in which the one or more corrugations of the first tube run.

Embodiment 4. The device of any one of Embodiments 1-3, wherein the corrugated region of the first tube comprises a helical corrugation.

Embodiment 5. The device of any one of Embodiments 1-3, wherein the corrugated region of the first tube comprises one or more corrugations defining a corrugation axis defined along the portion of the corrugation that is at the maximum distance measured radially outward from the major axis of the first tube, the corrugation axis being at a corrugation angle that is non-perpendicular to the major axis of the first tube.

Embodiment 6. The device of any one of Embodiments 1-3, wherein the corrugated region of the first tube comprises corrugations having different widths, pitches, or both.

Embodiment 7. The device of Embodiment 2, wherein the corrugated region of the first tube comprises a helical corrugation.

Embodiment 8. The device of Embodiment 2, wherein the corrugated region of the first tube comprises one or more corrugations defining a corrugation axis defined along the portion of the corrugation that is at the maximum distance measured radially outward from the major axis of the first tube, the corrugation axis being at a corrugation angle that is non-perpendicular to the major axis of the first tube.

Embodiment 9. The device of Embodiment 2, wherein the corrugated region of the first tube comprises corrugations having different widths, pitches, or both.

Embodiment 10. The device of any one of Embodiments 1-9, further comprising a vent defined by the second tube and the first tube communicating with the sealed insulating space to provide an exit pathway for gas molecules from the space, the vent being sealable for maintaining a vacuum within the sealed insulating space following evacuation of gas molecules through the vent, the distance between the first and second walls being variable in a portion of the insulating space adjacent the vent such that gas molecules within the insulating space are directed towards the vent by the variable-distance portion of the first and second walls during the evacuation of the insulating space, the directing of the gas molecules by the variable-distance portion of the first and second walls imparting to the gas molecules a greater probability of egress from the insulating space than ingress.

Embodiment 11. The device of any one of Embodiments 1-10, further comprising a spacer material disposed in the sealed insulating space.

Embodiment 12. The device of Embodiment 11, wherein the spacer material defines one or more surface features that is oriented perpendicular to the corrugations of the first tube.

Embodiment 13. The device of any one of Embodiments 11-12, wherein the spacer material defines one or more surface features that is oriented perpendicular to the corrugations of the second tube.

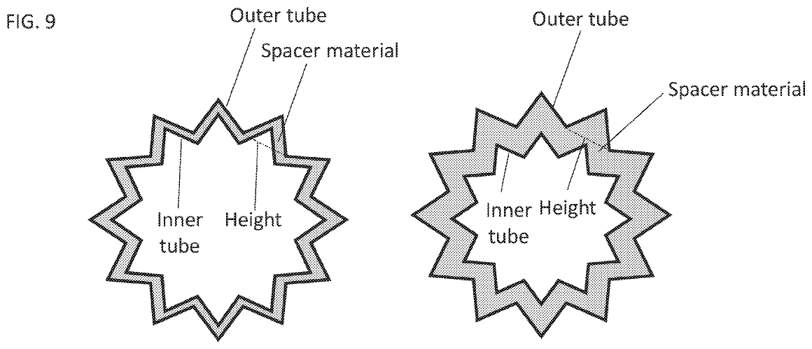

Spacer material can be of a thickness that the spacer material is disposed within the grooves of corrugations without extending beyond the corrugations. Such a configuration is shown in the left panel of FIG. 9, which panel shows a spacer material that lies within the grooves of the corrugations of the outer tube, but which material is not of a thickness that extends beyond the height of the corrugations or that “stands proud” of the corrugations. An alternative embodiment is shown in FIG. 9, right panel, which panel shows a spacer material that lies within the grooves of the corrugations of the outer tube, but which material is of a thickness that extends beyond the height of the corrugations so as to “stand proud” of the corrugations.

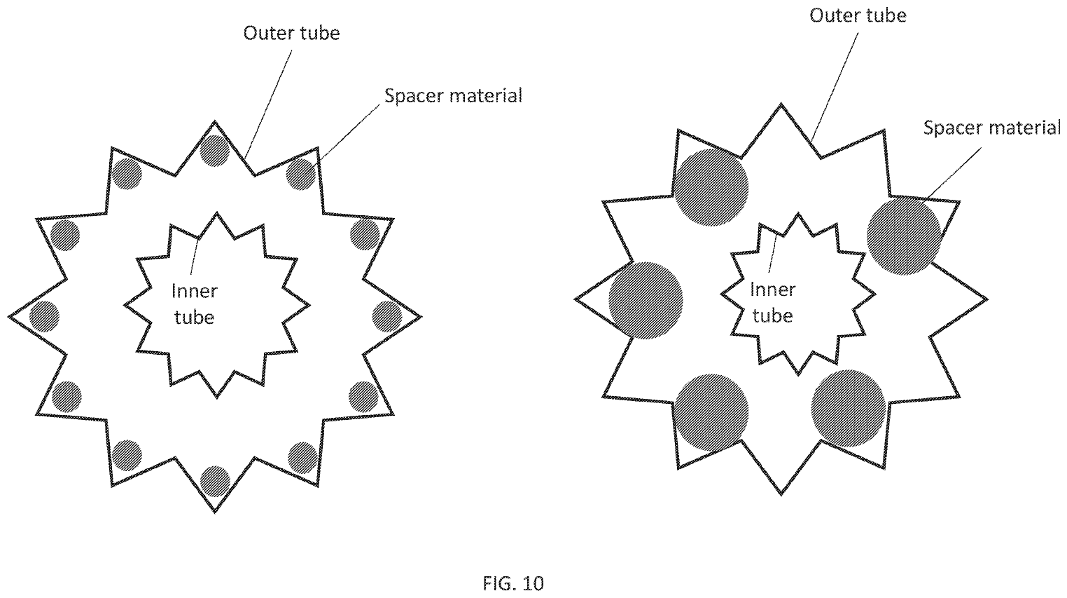

Another embodiment is shown in FIG. 10, which figure shows (left panel) a spacer material that can be present as a thread or other form that is disposed within the grooves of the corrugations of the outer tube while remaining within those grooves and not exceeding the height of the grooves. The left panel of FIG. 10 provides an embodiment in which the spacer material is of a thickness that the material can be disposed within the grooves but also extends beyond or “stands proud” of the height of the corrugations.

Embodiment 14. The device of any one of Embodiments 11-13, wherein the spacer material comprises a multi-layer insulation.

Embodiment 15. The device of any one of Embodiments 11-13, wherein the spacer material comprises a thread or a sheet.

Embodiment 16. The device of any one of Embodiments 11-15, further comprising a heating element configured to effect heating of a material disposed within the second tube.

Embodiment 17. The device of Embodiment 16, wherein the heating element is a resistance heating element.

Embodiment 18. The device of Embodiment 17, wherein the heating element is moveable.

Embodiment 19. The device of any one of Embodiments 11-15, further comprising at least one susceptor susceptible to inductive heating and at least one heating coil configured to give rise to inductive heating of the susceptor.

Embodiment 20. The device of Embodiment 19, wherein at least one of the at least one susceptor and the at least one heating coil is moveable relative to the other.

Embodiment 21. The device of Embodiment 19, wherein the susceptor is oriented essentially parallel with the major axis of the second tube.

Embodiment 22. The device of Embodiment 19, wherein the susceptor is oriented essentially perpendicular to the major axis of the second tube.

Embodiment 23. The device of Embodiment 19, wherein the susceptor is oriented at an angle of from about 1 to about 89 degrees to the major axis of the second tube.

A susceptor can be angled at, e.g., from about 1 to about 89 degrees to the major axis of the second tube, or from about 5 to about 85 degrees to the major axis of the second tube, or from about 10 to about 80 to the major axis of the second tube, or from about 15 to about 75 degrees to the major axis of the second tube, or from about 20 to about 70 degrees to the major axis of the second tube, or from about 25 to about 65 degrees to the major axis of the second tube, or from about 30 to about 60 degrees to the major axis of the second tube, or from about 35 to about 55 degrees to the major axis of the second tube, or from about 40 to about 50 degrees to the major axis of the second tube, or even at about degrees to the major axis of the second tube.

Without being bound to any particular theory or embodiment, a device can comprise a plurality of susceptors (or other heaters, such as resistive heaters) that are inserted or withdrawn from the interior of the second tube. This allows for user control over the temperature of a fluid that is communicated past the susceptors or heaters. As described elsewhere here, a susceptor or a heater can be individually addressable,

Embodiment 24. The device according to any one of Embodiments 1-23, wherein the second tube defines a first flow path region extending within the second tube and a second flow path region extending within the second tube, the first flow path region and the second flow path region being at an angle θ1 to one another.

Embodiment 25. The device of Embodiment 25, wherein 01 is from about 1 to about 180 degrees.

Embodiment 26. The device of Embodiment 25, wherein 01 is from about 30 to about 90 degrees.

Embodiment 27. The device according to any one of Embodiments 1-26, further comprising a third tube, the third tube comprising a lumen in fluid communication with the second tube, and the device defining a sealed insulating space of reduced pressure between the first tube and the third tube. One can, e.g., communicate a fluid through the second tube, the third tube, or both.

The second and third tubes can be joined directly together; the second and third tube can also be joined by way of a fitting, such as an elbow or other such fitting. In some embodiments, the sealed insulating space of reduced pressure between the first tube and the third tube is the same is or is in fluid communication with the sealed insulating space of reduced pressure between the first tube and the second tube.

Embodiment 28. The device of claim 27, wherein the second tube defines a first flow path extending within the second tube, wherein the third tube defines a flow path region extending within the third tube, the flow path region of the first tube and the flow path region of the second tube being at an angle θ1 to one another.

Embodiment 29. The device of Embodiment 28, wherein 01 is from about 1 to about 180 degrees.

Embodiment 30. The device of Embodiment 29, wherein 01 is from about 30 to about 90 degrees.

Embodiment 31. A method, comprising bending a device according to any one of Embodiments 1-30.

Embodiment 32. A method, comprising: with a device according to any one of Embodiments 1-30, communicating a fluid within the second tube.

Embodiment 33. A method, comprising: with a device according to any one of Embodiments 1-30, communicating a fluid exterior to the first tube.

1. A thermally-insulated device, comprising:
a first tube,
the first tube defining a major axis,
the first tube comprising a corrugated region that includes one or more corrugations,
(a) the one or more corrugations defining a corrugation axis defined along the portion of the corrugation that is at the maximum distance measured radially outward from the major axis of the first tube, the corrugation axis being at a corrugation angle that is non-perpendicular to the major axis of the first tube, or
(b) the corrugated region comprising a helical corrugation; and
a second tube,
the second tube having a major axis and the second tube being disposed within the first tube, and
the second tube and first tube defining a sealed insulating space of reduced pressure therebetween.

2. The device of claim 1, wherein the second tube comprises a corrugated region that includes one or more corrugations,
(a) the one or more corrugations defining a corrugation axis defined along the portion of the corrugation that is at the maximum distance measured radially outward from the major axis of the second tube, the corrugation axis being at a corrugation angle that is non-perpendicular to the major axis of the second tube, or
(b) the corrugated region of the second tube comprising a helical corrugation

3. The device of claim 2, wherein the one or more corrugations of the second tube run in a direction different from a direction in which the one or more corrugations of the first tube run.

4. The device of claim 1, wherein the corrugated region of the first tube comprises a helical corrugation.

5. The device of claim 1, wherein the corrugated region of the first tube comprises one or more corrugations defining a corrugation axis defined along the portion of the corrugation that is at the maximum distance measured radially outward from the major axis of the first tube, the corrugation axis being at a corrugation angle that is non-perpendicular to the major axis of the first tube.

6. The device of claim 1, wherein the corrugated region of the first tube comprises corrugations having different widths, pitches, or both.

7. The device of claim 2, wherein the corrugated region of the first tube comprises a helical corrugation.

8. The device of claim 2, wherein the corrugated region of the first tube comprises one or more corrugations defining a corrugation axis defined along the portion of the corrugation that is at the maximum distance measured radially outward from the major axis of the first tube, the corrugation axis being at a corrugation angle that is non-perpendicular to the major axis of the first tube.

9. The device of claim 2, wherein the corrugated region of the first tube comprises corrugations having different widths, pitches, or both.

10. The device of claim 1, further comprising a vent defined by the second tube and the first tube communicating with the sealed insulating space to provide an exit pathway for gas molecules from the space, the vent being sealable for maintaining a vacuum within the sealed insulating space following evacuation of gas molecules through the vent, the distance between the first and second walls being variable in a portion of the insulating space adjacent the vent such that gas molecules within the insulating space are directed towards the vent by the variable-distance portion of the first and second walls during the evacuation of the insulating space, the directing of the gas molecules by the variable-distance portion of the first and second walls imparting to the gas molecules a greater probability of egress from the insulating space than ingress.

11. The device of claim 1, further comprising a spacer material disposed in the sealed insulating space.

12. The device of claim 11, wherein the spacer material defines one or more surface features that is oriented perpendicular to the corrugations of the first tube.

13. The device of claim 11, wherein the spacer material defines one or more surface features that is oriented perpendicular to the corrugations of the second tube.

14. The device of claim 11, wherein the spacer material comprises a multi-layer insulation.

15. The device of claim 11, wherein the spacer material comprises a thread or a sheet.

16. The device of claim 11, further comprising a heating element configured to effect heating of a material disposed within the second tube.

17. The device of claim 16, wherein the heating element is a resistance heating element.

18. The device of claim 17, wherein the heating element is moveable.

19. The device of claim 11, further comprising at least one susceptor susceptible to inductive heating and at least one heating coil configured to give rise to inductive heating of the susceptor.

20. The device of claim 19, wherein (a) at least one of the at least one susceptor and the at least one heating coil is moveable relative to the other (b) wherein the susceptor is oriented essentially parallel with the major axis of the second tube, (c) wherein the susceptor is oriented essentially perpendicular to the major axis of the second tube, (d) wherein the susceptor is oriented at an angle of from about 1 to about 89 degrees to the major axis of the second tube, or any combination of (a), (b), (c), (d), and (e).

21. (canceled)

22. (canceled)

23. (canceled)

24. The device according to claim 1, wherein the second tube defines a first flow path region extending within the second tube and a second flow path region extending within the second tube, the first flow path region and the second flow path region being at an angle θ1 to one another.

25. The device of claim 24, wherein 01 is from about 1 to about 180 degrees.

26. The device of claim 25, wherein 01 is from about 30 to about 90 degrees.

27. The device according to claim 1, further comprising a third tube, the third tube comprising a lumen in fluid communication with the second tube, and the device defining a sealed insulating space of reduced pressure between the first tube and the third tube. The second and third tubes can be joined directly together; the second and third tube can also be joined by way of a fitting, such as an elbow or other such fitting.

28. The device of claim 27, wherein the second tube defines a first flow path extending within the second tube, wherein the third tube defines a flow path region extending within the third tube, the flow path region of the first tube and the flow path region of the second tube being at an angle θ1 to one another.

29. The device of claim 28, wherein 01 is from about 1 to about 180 degrees.

30. The device of claim 29, wherein 01 is from about 30 to about 90 degrees.

31. A method, comprising bending a device according to claim 1.

32. A method, comprising: with a device according to claim 1, communicating a fluid within the second tube.

33. A method, comprising: with a device according to claim 1, communicating a fluid exterior to the first tube.
